(19) United States
(12) Patent Application Publication
(10) Pub. No.: US 2013/0178747 A1
(43) Pub. Date: Jul. 11, 2013

(54) BREAST CANCER PRECURSOR EVALUATION SYSTEM AND PROACTIVE BREAST WELLNESS PROGRAM

(71) Applicant: Peter J. Muran, Arroyo Grande, CA (US)

(72) Inventor: Peter J. Muran, Arroyo Grande, CA (US)

(21) Appl. No.: 13/347,403

(22) Filed: Jan. 10, 2012

(51) Int. Cl. A61B 6/00

(52) U.S. Cl. 600/474

(57) ABSTRACT

A breast cancer precursor evaluation system and a proactive breast wellness program is disclosed. The evaluation system combines a DNA oxidative stress and/or estrogen-DNA adduct screening test and a thermography screening test to provide an indication of whether the patient is developing or at a significantly higher risk for breast cancer. Moreover, the breast wellness program has components related to vascular health, immune balance, hormone balance, healthy food intake, elimination of toxins and waste and routine monitoring.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

The various aspects disclosed herein relate to a precursor evaluation for breast cancer and a proactive breast cancer wellness treatment program.

The primary medical means of addressing breast cancer is through early detection. Contemporary thought is that early detection increases the chance of survival. The current state of the art method of detecting breast cancer is through a mammogram screening test. Unfortunately, the mammogram screening test is not an accurate test. There are many false negative results. Also, once a cancer tumor is detected on a mammogram screening test, the cancer cells have typically been developing for the past five (5) to ten (10) years. As such, breast cancer may not be detected or evident until it is too late. Moreover, the mammogram screening test requires compression of the breasts which is uncomfortable and radiation which also may increase the risk of developing breast cancer. Accordingly, if the patient has regular yearly mammograms, the patient's risk for developing breast cancer may also increase.

In studying breast cancer, the medical profession has come to broadly view estrogen as the primary cause of breast cancer. Accordingly, the medical profession routinely recommends medication to reduce or control estrogen levels of the patient or surgery to remove ovaries to eliminate production of estrogen. Unfortunately, removal or control of estrogen levels in the patient's body affects the patient's emotional and physiological health. The estrogen hormone is what makes the female feel like a female and be a female. If estrogen is the cause of breast cancer, then there would be a steady increase in breast cancer corresponding to the population increase. However, the incidence of breast cancer has risen 171% while the female population has risen only by 29% during the same time period.

Accordingly, there is a need in the art for an improved breast cancer precursor and breast wellness program.

### BRIEF SUMMARY

The various aspects discussed herein address the needs discussed above, discussed below and those that are known in the art.

The treatment discussed herein relates to an improved breast cancer precursor evaluation system and a proactive breast wellness program. The evaluation system includes a DNA oxidative stress screening, test which screens the patient for an abnormally high indication of DNA oxidative stress, and/or an estrogen-DNA adduct test which measures the amount of depurinating estrogen-DNA adducts caused by the DNA oxidative stress. This test can be tested via urine or other bodily fluids of the patient. Elevated levels of DNA oxidative stress can cause DNA damage leading to elevated levels of estrogen-DNA adducts, which can contribute to the mutantgenecity of the cell and lead to cancer formation. The evaluation system also includes and combines the DNA oxidative stress and/or estrogen-DNA adduct test with a thermography screening test. The thermography screening test may be a single thermal image or a series of thermal images which detects significant changes in the thermal signature of the breasts of the patient. When these two screening tests (i.e., (1) DNA oxidative stress and/or estrogen-DNA adduct screening test and (2) thermography screening test indicate a higher risk of breast cancer for the patient (i.e., positive), the patient may be prescribed a proactive breast wellness regimen which includes at least one of the following components related to improving the patient's vascular health including stimulation of lymphatic flow, digestion sufficiency, immune balance, hormone balance, food intake, elimination of toxins and waste, and inner balance. The breast wellness regimen is coupled with routine monitoring of the patient's breasts via thermography screening tests, the DNA oxidative stress and/or estrogen-DNA adduct screen test to track effectiveness of the breast wellness regimen. Additionally, or alternatively to the other breast wellness regimen components, the patient may be provided supplements targeted for individualized detoxification and nutrient requirements in the estrogen detoxification pathways.

More particularly, a method for identifying a patient with an increased propensity to breast cancer and implementing a responsive treatment is disclosed. The method may comprise the steps of conducting at least one DNA oxidative stress and/or estrogen-DNA adduct screening test on a patient to detect a first precursor biomarker for detection of breast cancer risk; conducting a single or series of thermography screening tests on the patient to detect a second precursor biomarker for detection of breast cancer risk; based on the first and second precursor biomarkers, diagnosing whether the patient is at a higher risk for breast cancer wherein the patient is at the higher risk for breast cancer when the first and second precursor biomarkers are positive; and recommending a breast wellness regimen to the patient based on results of the tests for the first and second biomarkers.

In the method, the step of conducting the DNA oxidative stress and/or estrogen-DNA adduct screening test may detect a level of DNA oxidative stress and/or estrogen-DNA adduct. More particularly, the DNA oxidative stress or estrogen-DNA adduct screening test may detect abnormally high levels of DNA oxidative stress markers and/or high ratios of depurinating estrogen-DNA adducts to their respective estrogen metabolites and conjugates and a measurable oxidative stress environment which can lead to the formation of the depurinating estrogen-DNA adducts as is known in the art and described in greater detail herein. Exemplary markers of DNA oxidative stress include high levels of superoxide dismutase (SOD1 and SOD2), nuclear factor kappa B (NFkB), 8-hydroxy-deoxyguaninosine, and glutathione, reduced and oxidized. The level of the DNA oxidative stress and/or the level of depurinating estrogen-DNA adducts may be the first biomarker and may be positive when the ratios are significantly high compared to a control group of females with a low risk for breast cancer.

In the method, the step of conducting a single or series of thermography screening tests may comprise the steps of conducting thermography screening test on the patient to acquire a heat map of the breasts of the patient indicating areas of normal to inflamed breast tissue. The thermographic heat maps are indicative of changes in metabolic activity in the breast tissue as evidenced by normal to inflamed breast tissue and blood flow variations in heat patterns. Initial thermographic heat maps may show remarkable patterns of inflammation to be further investigated. As well, thermograms over a period of time may show significant changes in an individual's heat and blood flow patterns that may be indications of developing inflammation. Successive thermography screening tests on the patient can be used to monitor improved, stable or declining states of inflammation as compared to prior thermographic screenings. A significant change in inflammation is the second precursor biomarker and is positive when there is a significant indication of abnormal heat patterns.

The DNA oxidative stress and/or estrogen-DNA adduct screening test may be conducted if the second biomarker is positive. Alternatively, the thermography screening tests may be performed if the first biomarker is positive. In a further alternative, both the DNA oxidative stress and/or estrogen-DNA adduct screening test and the thermography screening test may be conducted regardless of the outcome of the other test.

The steps of conducting the DNA oxidative stress and/or estrogen-DNA adduct screening test and/or the steps of conducting a single or series of thermography screening tests may be performed at regular intervals as the patient conducts the breast wellness regimen.

The breast wellness regimen may include the step of inducing lymphatic fluid flow through the patient's lymphatic system. The inducing step may include one or more of regular physical exercise, consumption of water, fitted clothing and lymphatic breast massage.

The breast wellness regimen may include steps to improve one or more of the patient's immune balance, hormone balance, food intake, and toxin and waste elimination. Additionally, or alternatively, to the other breast wellness regiment components, in order to improve a patient's estrogen detoxification pathways, one or more supplements known to aid in estrogen detoxification may be taken by the patient. If after a period of time (for example, three months) the patient has not seen an improvement in the results of one or more of the thermography screening tests, DNA oxidative stress and/or estrogen-DNA adduct screening tests, the patient may undergo a urine metabolic profile, followed by directed genetic testing, in an attempt to discover genetic variants in the patient that may be causing an underproduction in enzymes utilized in the estrogen deactivation pathways. If a genetic deficiency is discovered, it may be corrected by providing particular supplementation to the patient.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a flow chart of an improved breast cancer precursor evaluation system and proactive breast wellness program;

FIG. 2 illustrates a base line thermography temperature signature of breasts of a patient;

FIG. 3 illustrates a changed thermography temperature signature of the breasts of the patient shown in FIG. 2;

FIG. 4 illustrates a first technique to induce circulation of lymphatic fluid in a breast portion of a lymphatic system of a patient;

FIG. 5 illustrates a second technique to induce circulation of lymphatic fluid in the breast portion of the lymphatic system of the patient;

FIG. 6 illustrates a third technique to induce circulation of lymphatic fluid in the breast portion of the lymphatic system of the patient;

FIG. 7 illustrates a fourth technique to induce circulation of lymphatic fluid in the breast portion of the lymphatic system of the patient; and

FIG. 8 is a flowchart illustrating portions of estrogen metabolism as influenced by DNA oxidative stress and portions of the pathway amenable to corrections so as to reduce DNA damage and subsequent cancer, as per an increase in the estrogen-DNA adduct production.

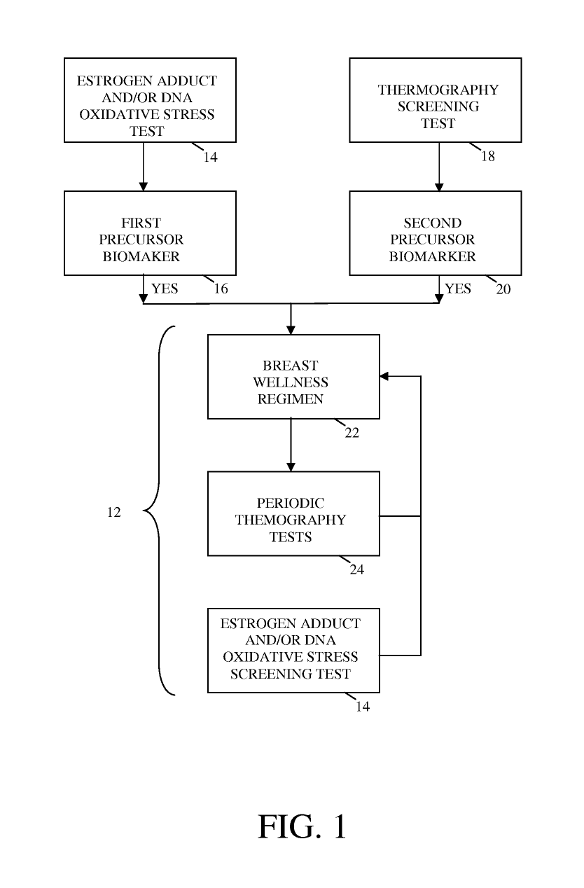

### DETAILED DESCRIPTION

Referring now to the drawings, a breast cancer precursor evaluation system 10 and breast wellness program 12 are disclosed. The system 10 and program 12 combine a DNA oxidative stress and/or estrogen-DNA adduct screening test 14 to detect a first precursor biomarker 16 to breast cancer, namely, levels of DNA oxidative stress markers and/or specific depurinating estrogen-DNA adducts (e.g., estrogen-nucleoside, estrogen-guanine and/or estrogen-mercapturate adducts) and a breast thermography screening test 18 to detect a second precursor biomarker 20 to breast cancer. These two tests 14, 18 are used to identify and monitor risk for the development of breast cancer in a patient. Based on the results of these two tests, a breast wellness program 12 may be prescribed to the patient to actively manage the risk of developing breast cancer. In particular, the breast wellness program 12 shifts DNA expression to reduce the risk for the development of breast cancer in the patient. As a result, breast cancer may be prevented instead of merely being detected as early as possible. Furthermore, in addition to or in place of the other components of the breast wellness program, supplementation may be prescribed to the patient to increase the support of estrogen detoxification pathways and reduce DNA oxidative stress.

The medical profession has come to believe that the estrogen hormone causes breast cancer. As a result, the medical profession has recommended some patients with a high risk of breast cancer to remove one or both breasts, remove ovaries which produce the estrogen or take medication to control the level of estrogen in the patient's system. However, estrogen in its natural state, unmodified, does not cause cancer. Estrogen works with a woman's body to provide various benefits including but not limited to psychological and neurological, signaling of other hormones, muscular and skeletal, immunological, cardiovascular health and prevention of breast cancer among some other four hundred (400) different functions. Estrogen is important to the woman's body.

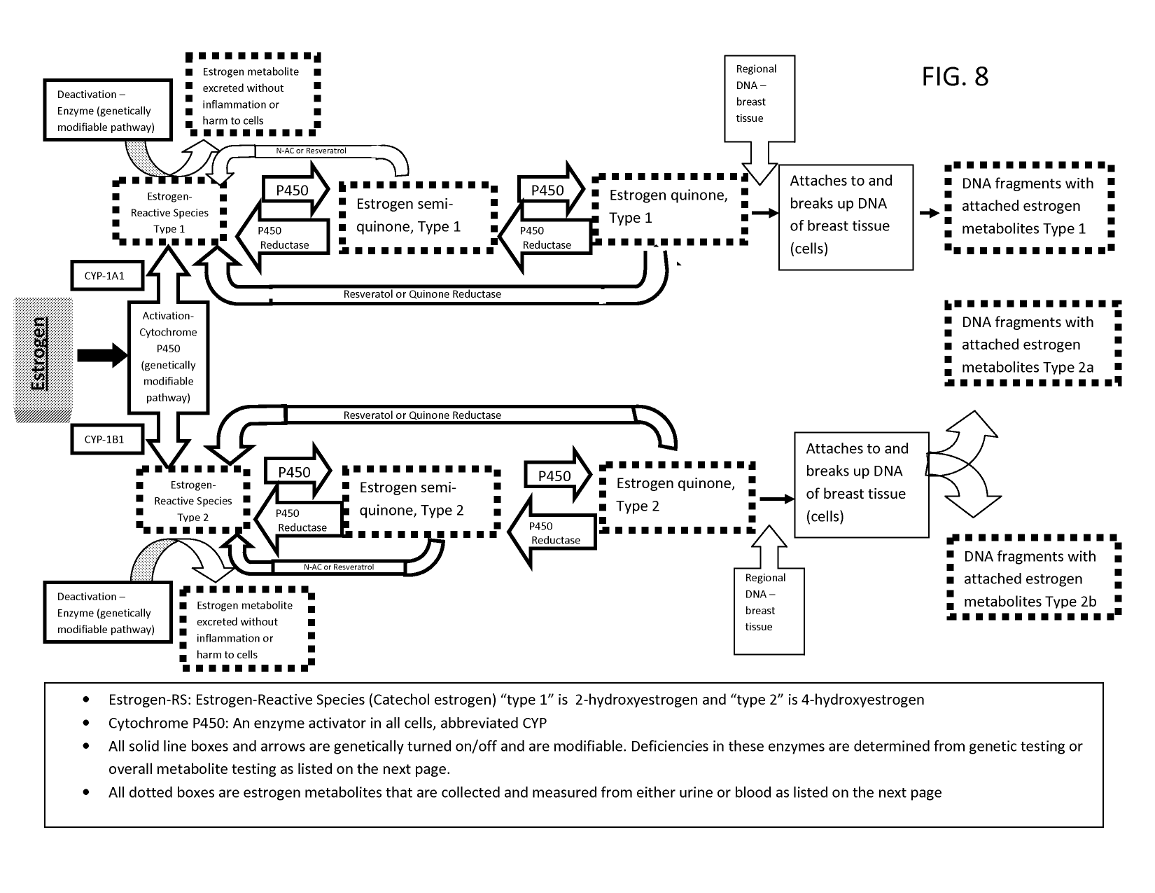

Three types of estrogen are produced by the woman's body, namely, estrone, estradiol and estriol. As used herein, estrogen encompasses all three types of estrogen. After estrogen is produced, estrogen enters cells and communicates with the DNA of the cell to produce building blocks of the female bodies such as coding, selectivity and formation of every cell from our brain, to our heart, to the blood pumping throughout the entire body. Hence, estrogen is important and vital to proper functioning of the female's body. FIG. 8 describes the possible detoxification pathways and junctures where intervention can occur to positively influence DNA expression further detailed in the following.

Estrogen leaves the cell through a two-step process, namely, an activation step and a deactivation step. In the first step, damaging chemicals known as estrogen-reactive species are activated. In the second step, these damaging chemicals are deactivated with antioxidants and safely eliminated. To reduce the risk of breast cancer, both the activation step and deactivation step are addressed. The first activation step may produce two different types of estrogen-reactive species (“estrogen-RS”). However, one type or species of oxidants has a higher risk for causing breast cancer. The breast wellness program facilitates or promotes production of the lower risk oxidants. The second deactivation step may be blocked by insufficient nutrients and debris of metabolic waste and harmful toxins, which increases the DNA oxidative stress profile. If the second step is hampered or blocked, then the estrogen-RS may evolve into a more powerful estrogen-reactive species, furthering the increase in the DNA oxidative stress profile. As the estrogen-RS becomes more potent, the body's immune system responds by releasing higher levels of NFkB which is an inflammatory precursor biomarker to cancer. It is this inflammation being detected through the breast thermography screening test. If the estrogen-RS is not deactivated, then such highly potentiated estrogen-reactive species will continue to become so highly reactive that it attaches to DNA for deactivation, but in doing so, the DNA is damaged. If the DNA is not repaired, this damaged DNA leads to a mutagenic cell, which then replicates a lineage of mutant cells leading to a cluster of cancer cells. Throughout the process, the body provides multiple opportunities to safely deactivate the estrogen-reactive species and reduce the DNA oxidative stress. Through the breast wellness program, the environment within the person's body to safely reduce the DNA oxidative stress and deactivate the estrogen-reactive species is promoted. The breast wellness program 12 shifts the DNA expression to reduce the risk for the development of breast cancer. Moreover, when the estrogen-reactive species reacts with DNA, specific depurinating estrogen-DNA adducts identified above may be formed which are also a precursor biomarker to breast cancer. These specific estrogen-DNA adducts are generated by difficulty in the cells to properly detoxify estrogen and have been shown to lead to the development of breast cancer. It is these specific depurinating estrogen-DNA adducts being detected through the urine screening test.

Some women may have genetic alterations which prevent the effective accomplishment of steps one and two of detoxification. Examples include BRCA1 and BRCA2 genes; altered or missing enzymes required for step 1 and/or 2. If indicated, further genetic testing may be required to “patch” the capacity for detoxification. This patch would include individually identified natural nutrients such as, but not limited to, tetrahydrofolate, folate, folic acid (and its derivatives), phosphatidyl serine complex, phosphatidyl choline, cobalamin (vitamin B12), intrinsic factor, pyridoxine (vitamin B6 or its derivatives), thiamine (vitamin B1), biotin (vitamin B7), niacin (vitamin B3), riboflavin (vitamin B2), n-acetyl cysteine (NAC), sulphorophance, SAMe, RNA nucleotides, amino acids, glycine, GABA, CoQ10, L-Carnitine (or its derivatives), alpha lipoic acid, resveratrol, glutathione (or its derivatives), DIM (diindolymethane), TMG and omega 3 fatty acids.

In the breast cancer precursor evaluation system, a medical professional may order (e.g., prescribe, recommend, etc.) a female patient to undergo a DNA oxidative stress and/or an estrogen-DNA adduct screening test 14 which detects the specific level of DNA oxidative stress and/or depurinating estrogen-DNA adducts 16 mentioned above. If the ratio of these specific level of DNA oxidative stress and/or depurinating estrogen-DNA adducts 16 to respective metabolites or conjugates are statistically high compared to a control group of females with a low risk to breast cancer, then the results of the DNA oxidative stress and/or estrogen-DNA adduct screening test will indicate that the patient being tested is at a higher risk for breast cancer. Alternatively, if the absolute values of the specific levels of DNA oxidative stress and/or depurinating estrogen-DNA adducts mentioned above is abnormally high then the results of the DNA oxidative stress and/or estrogen-DNA adduct screening test 14 will indicate that the patient being tested is at a higher risk for breast cancer.

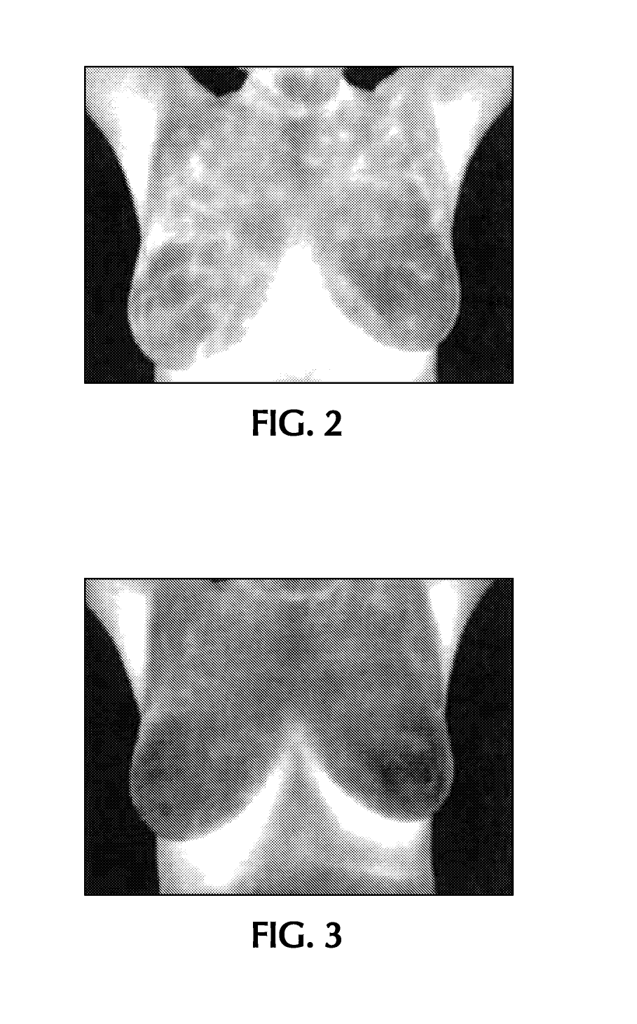

In conjunction with the DNA oxidative stress and/or estrogen-DNA adduct screening test 14, the medical professional may order the patient 18 to undergo breast thermography screening testing to detect inflammation of the breast tissue and monitor the efficacy of the breast wellness program through a single thermogram or a series of time spaced thermograms. If a series of thermograms are taken, then the time period between tests may be any number of months up to 1 year. The thermography screening test detects differences in inflammation in the patient's breast over a period of time. If the level of inflammation increases over a period of time, then the patient's breast or localized inflamed areas of the patient's breasts may be detected on the thermogram or heat map of the patient's breast. The thermogram shown in FIG. 3 has a significantly different thermal map of the patient's breasts compared to FIG. 2. Thermography or medical infrared imaging has infrared sensors which detect heat emitted from the body. The sensors detect heat emitted from the breasts of the patient. The sensed data is compiled for quantitative computerized analysis which creates the images shown in FIGS. 2 and 3. Each patient has a unique thermovascular temperature pattern which is monitored over time. The differences in the thermograms taken at two different times reflect an underlying physiological process, such as increased inflammation and/or blood flow related to tumor growth (angiogenesis). It is these changes that provide an indication of inflammatory metabolic activity which can be early indications of risk for the development of breast cancer; the presence of cancer or the early development of cancerous cells. These changes are caused by increase level of DNA oxidative stress and/or improper deactivation of the estrogen-RS. Although the thermography screenings may be done over a period of time, a single thermography screening test may detect significant changes which indicate a higher risk of cancer.

The benefits of the thermography screening test 18 are that this test 18 does not involve radiation or compression. Thermography is non invasive and does not require compression of the breasts as in mammography. Thermography is effective for women of all ages, all breast types and breast densities (including breast implants and reductions). Thermography can detect changes which may indicate breast cancer earlier than mammography. Thermography does not pinpoint the exact spot of suspicion and does not diagnose cancer. Rather, thermography can detect a pathologic state of the breast up to ten (10) years before a cancerous tumor is found by other methods known in the art. Thermography does not have any side effects and may be conducted as often as necessary.

The following individual breast wellness program 12 may comprise a breast wellness regimen 22 which is followed by periodic thermography tests 24 to track effectiveness of the breast wellness regimen 22 and progress of reducing the second precursor biomarker to breast cancer. Moreover, the DNA oxidative stress and/or estrogen-DNA adduct screening test 14 may be periodically conducted to track effectiveness of the breast wellness regimen and progress of reducing the first precursor biomarker to breast cancer. One or more components of the breast wellness regimen 22 may be prescribed or recommended by a medical professional (e.g., doctor, etc.). The breast wellness regimen 22 may address one or more of the following components vascular health (including stimulation of lymphatic flow), hormone balance, digestion sufficiency, immune balance, healthy food intake, waste and toxin elimination and inner balance. These components work to safely deactivate the estrogen-RS. Simply put, it shifts the DNA expression to reduce the risk of development of breast cancer. It is designed to address areas in which one's choices with regard to these components can reduce risk of exposure and enhance the body's natural ability to express healthy DNA function expression. Once the estrogen is activated and converted into estrogen-RS, the body provides numerous opportunities for safe deactivation of the estrogen-RS. The breast wellness program 12 disclosed herein promotes the proper conditions within the patient's body to safely deactivate the estrogen-RS and a method to track effectiveness of the proactive efforts of the patient to reduce or prevent breast cancer.

Vascular health as used herein relates to the lymphatic system which impacts the creation of proper conditions within the patient's body to safely deactivate the estrogen-RS. An adult has about 10 quarts of lymphatic fluid compared to 5 quarts of blood. The difference between the lymphatic system and the cardiac system is that the lymphatic system does not have a dedicated pump (i.e., heart). Lymphatic flow through the lymphatic system is dependent upon movement and gravity. Congestion of the lymphatic system may occur which interferes with essential immune function and discharge of metabolic waste.

To improve the patient's vascular health, sufficient amounts of water should be consumed throughout the day to service the patient's body size. One may calculate the proper amount of water to consume by dividing the patient's weight by two (2) which represents the number of ounces of water needed by the patient. Other beverages such as carbonated soda and coffee do not count toward the water intake. If the patient consumes caffeinated drinks, then the amount of water intake should be increased above the calculated amount mentioned above. Moreover, the patient should drink a quart of water for every hour of exercise.

Physical exercise is also prescribed to improve the vascular health of the patient. Physical exercise involves muscular contraction which also circulates the patient's lymphatic fluid. Suitable physical exercises include and are not limited to tai chi, yoga, walking, biking, swimming or jogging. The patient should engage in physical exercise four (4) times a week for a minimum duration of twenty (20) minutes each occurrence. The patient's occupation should also be taken into consideration. If the patient's occupation is sedentary, then adjustments should be made to increase physical exercise through a change in job, career and occupation or readjusting one's schedule to increase physical exercise on the job.

Heat packs which provide concentrated heat may also be prescribed to the patient. The heat packs are applied to the breasts of the patient. By doing so, circulation of lymphatic fluid is stimulated. The heat packs may be castor oil packs.

Lymphatic fluid flow may be hampered by clothing. In particular, sometimes brassieres may constrict lymphatic out-flow causing localized congestion and inflammation. This compression may be accented if breast implants act as a solid mass with the breast tissue compressing between the implant and the brassieres. Accordingly, the patient should be fitted with the proper brassiere.

To further enhance lymphatic fluid flow, the patient may manually induce circulation of lymphatic fluid through lymphatic breast massage which mimics how lymphs move in the patient's body. The basics of lymphatic breast massage are to impart a pumping motion to the breast. Light pressure is applied to the breast. The breast is released gently allowing the breast to return to its normal position. Each move and return is counted as one pump or cycle. The key to lymphatic breast massage is to maintain constant flow of lymphatic fluid flow in the area being treated.

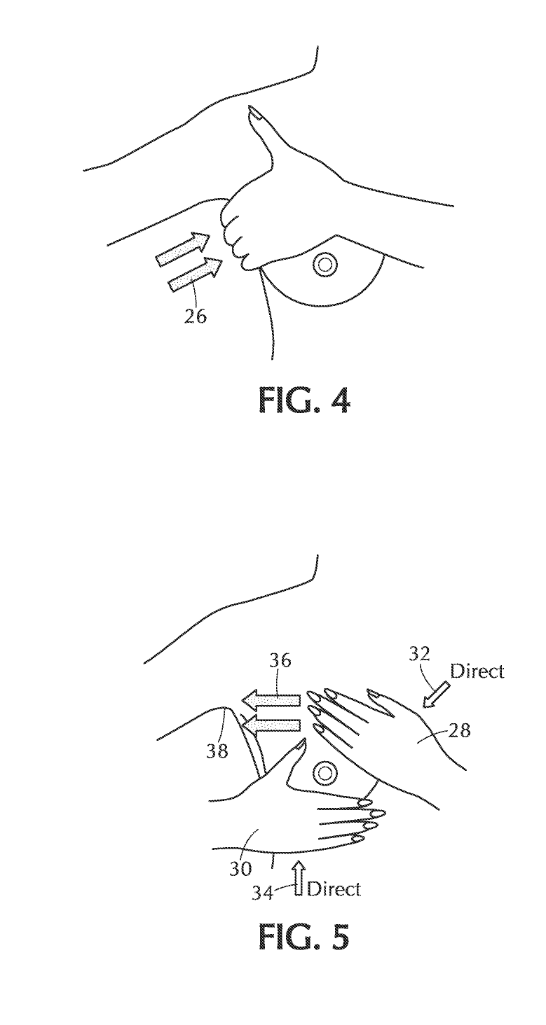

A first massage technique which is shown in FIG. 4 involves first placing one hand under the armpit and pushing upward and inward as shown by directional arrow 26. The movement should penetrate deeply into the armpit. Pump upwards toward the breast bone and release. It is recommended that this movement be conducted ten (10) to twenty (20) times.

A second massage technique which is shown in FIG. 5 involves firmly holding the entire breast and moving the entire breast upward toward the armpit. As shown, the left and right hands 28, 30 press the breast in direction of arrows 32, 34. Thereafter, the both hands 28, 30 are moved in direction of arrow 36 toward the armpit 38. If the breasts are too large, then more than one hand position may be required. This movement should be performed about ten (10) times.

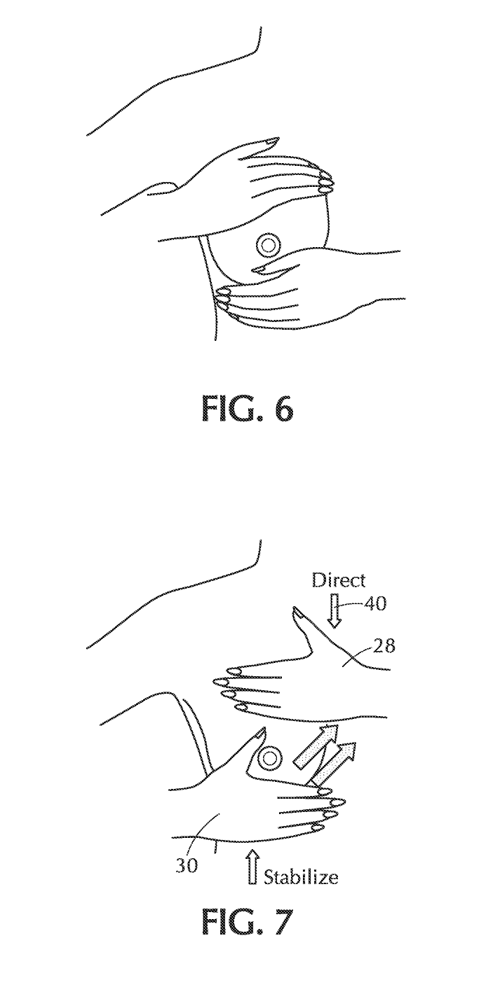

A third massage technique which is shown in FIG. 6 involves firmly holding the breast and pumping directly inward toward the chest wall. This movement should be performed about five times.

A fourth massage technique which is shown in FIG. 7 involves holding the breast firmly and pumping the inner quadrant of the breast up toward the patient's neck. The right hand stabilizes the breast while the left hand 28 presses down on the breast as shown by arrow 40. After the left and right hands 28, 30 are in place, the both hands 28, 30 shift the breast in the direction of arrow 42.

The four massage techniques were shown and described in relation to the patient's right breast. The same techniques may be applied to the patient's left breast in an opposite manner.

The lymphatic system has a one way valve. As such, the fluid cannot be pumped backward by mistake and cause harm. Additionally, when performing the above massage techniques, focus on creating movement. Direction of the lymphatic fluid is not an issue due to the one way valve discussed above. The initial movement should involve some pressure. When pressure is released, let the breast return to its normal position. This becomes the pumping action. Movement of the breasts and releasing to allow the breast to return to its normal position mimics and enhances natural movement of the lymphatic system and circulation of the lymphatic fluid.

To improve the patient's immune balance, sleep is required. Over stimulation or under stimulation of the immune system may impact the health of breast tissue. Over stimulation of the immune system may be expressed as inflammation which may show up as an area of concern on a thermogram. Under stimulation may cause the body not to provide an immunological response to the colonization of cancer cells. Restful, quality sleep provides the patient's immune system time to rest and strengthen itself. This includes sufficient time for sleep but also proper preparation for sleep to induce release of melatonin. Turn off the television and computer. Dim the lights before sleeping which triggers production of melatonin. Also, engage in relaxing activity such as a warm bath, soft music and/or reading prior to sleep time. If the patient has problems falling asleep, staying asleep or feeling rested when awoken, then the patient may be referred to a sleep specialist to diagnose and resolve the problem so that the patient may resume normal sleep and provide the immune system the needed time to rest and strengthen.

Balanced hormones are also beneficial for a healthy breast. Hormone imbalance has been identified as a catalyst for breast disease. Hormone balance includes the sex hormones of estrogen, progesterone and testosterone and the directors of the hormone cascade, thyroid, adrenals and insulin. To regain balanced hormones, laboratory evaluation of the above hormones are clinically evaluated. By way of example, the laboratory evaluation may include but is not limited to a 24 hour urine collection to determine how these hormones are being metabolized. This test will allow the medical professional to diagnose whether the hormones are being metabolized toward cancerous cells or being safely eliminated from the patient's body. Based on the results of the urine test, a clinical treatment is prescribed to restore balance. The clinical treatment may utilize the functional medicine approach. By way of example and not limitation, to restore hormonal balance, dietary supplements may be taken by the patient to restore hormonal balance to the patient. These dietary supplements include but are not limited to iodine supplementation, DIM (Di-Indole Methane), and indole-3-carbinal.

Healthy food intake is crucial to facilitate healthy breasts. Food provides instructions for thousands of metabolic functions in all cells of the body. First, eliminate foods which contribute to inflammation and acidic pH, deplete nutrients and increase glucose levels. To do so may require elimination of all processed foods. Bleached white foods, packaged foods and invented foods do not contribute to healthy breasts but divert the body's natural resources and compromises the body's immune system. Furthermore, sugars and trans-fats should be reduced. Include in one's diet nutrient dense foods which minimize chemical exposure, decrease inflammation and balance pH. By way of example and not limitation, food should be in its natural form. Emphasis of food intake should be on foods which contains omega 3 fatty acids, antioxidants, bio-flavinoids and anti-inflammatory nutrients known to positively affect breast tissue health. Food sources include cold water fish, flaxseeds, colorful fruits and vegetables and tumeric spice. Consume healthy fats such as fatty wild fish like salmon, small tunas, sardines and anchovies. Also, consume cold pressed, unrefined oils such as flax, olive and coconut. Consume fruits and vegetables that are brightly colored and grown organically. Fruits and vegetables contain cancer fighting bioflavinoids, vitamin C and beta-carotene. Cabbage, broccoli, brussel sprouts, kohlrabi, mustard greens, turnip greens, cauliflower, kale and collard greens are high in phyto-chemicals which inhibit estrogen synthesis that is inflammatory. Each meal should be balanced. Two thirds vegetables and one third protein from an organic, sustainable source should be consumed. Eat fruit alone to aid digestion. Consume blueberries, cranberries, raspberries and grapes which have anthocyanin, and anti-tumor property. Garlic, ginger and tumeric which may have anti-cancer properties should be included in the patient's diet. Flax seeds may be added to salads and muffins to reduce disease related genetic expression. The optimal healthy diet is individualized to meet the personal nutrient needs and metabolic function.

Even if the right foods are being consumed by the patient, the patient's digestive system must absorb the beneficial components of the food. As such, an evaluation of the patient's digestive competence to absorb nutrients is performed. As part of the digestive evaluation, the availability of adequate digestive enzymes is determined as well as whether the patient is eliminating waste regularly. Based on the evaluation of the patient's digestive system, the medical professional may recommend to the patient digestive enzymes, HCL (i.e., hydrochloric acid) and/or vinegar to support optimal digestion. Moreover, an evaluation may be conducted on the patient to determine the patient's food intolerances such as gluten, cow, dairy, soy and citrus which may add to digestive incompetence and mal-absorption.

With respect to eliminating waste and toxins from the patient's body, the major organs of the body that accomplish this task are the lungs, skin, kidneys, liver and colon. If these systems are inefficient, then as a result, the patient may have limited aerobic capacity, skin rashes and hives, lack of hair and skin luster, sensitivity to odors, foggy thinking, frequent respiratory illness, intestinal gas and constipation. To facilitate efficient functioning of the body's organ that eliminate waste and toxins, water intake should be monitored and increased as discussed above. Water intake aids in circulation of lymphatic fluid and aids the kidneys to flush out the toxins. Dry brushing the skin and Epsom salt baths help the skin to release toxins as well as perspiring as a result of exercise. Evaluate liver function and resolve any problems under medical guidance. The healthy colon should eliminate waste every time food is consumed. If not, the medical professional may prescribe or recommend a treatment to resolve this issue. The patient should resolve this issue. Decrease daily exposure to toxins. Make sure that the patient's home is chemical free which reduces the load on the patient's body to process and eliminate the toxins and waste. The patient should reduce exposure to toxins and xeno-estrogens in the home environment which have been linked to development of breast cancer. The medical professional should also recommend or prescribe reduction or elimination of lifestyle choices that minimize chemical exposure such as elimination of smoking, food additives, pesticides, etc. For example, the medical professional may prescribe patches, gums, pills, etc. to reduce the urge to smoke, etc.

Inner balance is also recommended. Women invest much of their energy in nurturing and caring for others frequently without monitoring their own health and emotional well being. The patient is encouraged to develop a sense of control, belonging to a community and a commitment to purpose. By improving the patient's inner balance, the capacity of the patient to care for herself is enlarged. The patient is encouraged to think through life's purpose and to commit to such life's purpose as it fits the patient. The patient is encouraged to open up time for joy and contemplation. Moreover, the patient is encouraged to reach out to the community. The patient is also encouraged that through the various aspects disclosed herein that the patient has control over his/her health and life.

As is partially illustrated in FIG. 8, estrogen metabolic and detoxification pathways are well known in the art and may be improved by supplementation with enzymes involved in these pathways and/or other substances that aid in the detoxification process. For example, certain enzymes within the cytochrome P450 superfamily are known to be utilized in estrogen metabolism. These enzymes include those encoded by the CYP1A1, CYP2A6, CYP2E1, CYP1C19, CYP1B1, CYP2D6, CYP2C9, and CYP4A4 genes. Furthermore, other enzymes capable of conjugating and eliminating toxins include, for example, MAO A R297R (monamine oxidase A); ACAT 102 (acetyl coenzyme A acetyltransferase); ACE (angiotensin converting enzyme); MTHFR A1298C, C677T (methylenetetrahydrofolate reductase); MTR A2756G/MTrr A66G, H595Y, K350A, R415T, S257T, 11 (methionine synthase/methionine synthase reductase); COMT (catechol-O-methyltransferase); NAT1 (N-acetyltransferase 1); NAT2 (N-acetyltransferase 2); GSTM1 (glutathione S-transferase Mu 1); GSTT1 (glutathione S-transferase theta-1); GSTP1 (glutathione S-transferase P); SOD1 (superoxide dismutase 1); SOD2 (superoxide dismutase 2); and VDR (vitamin D receptor). A patient may be suffering from a deficiency in any of these enzymes, which may then lead to reduced estrogen detoxification, and thus a higher risk of developing breast cancer.

As such, in addition to, or alternatively to, the other components of the breast wellness regimen disclosed above, a patient may take specific nutrients in an effort to shift the DNA functional expressions of these enzymes in order to support the estrogen detoxification pathways. For example, a patient may be provided any, or all, of diindolylmethane, Nrf2 activators, SAMe (S-adenosyl methionine), TMG (trimethylglycine), resveratrol, NAC (N-acetylcysteine), folic acid, and glutathione, in a first supplemental plan. In one example, a patient may be given the above compounds for a period of three months, at which point the patient will again receive thermogram and/or testing. If the first and/or second biomarkers remain at an unsatisfactory level after the patient has undergone the first supplemental plan, the patient may proceed to individual metabolic profiling followed by DNA testing in attempt to discover the specific blocks in the patient's metabolic pathway. In particular, the patient may undergo a metabolic profile, as is well known in the art, to determine where the metabolic blockage occurs. Once an irregularity has been determined from the metabolic profile, directed genetic testing may be performed in order to determine whether the patient has a genetic variant, or SNP (single nucleotide polymorphism), thereby posing difficulty for the patient to make the necessary metabolic enzymes. Examples of these enzymes were discussed above. Once the cause of the metabolic pathway disturbance has been identified, it may be treated providing the patient with the missing enzyme, and/or supplements that support the production of the enzymes. Examples of these supplements include: tetrahydrofolate, folate, folic acid (and its derivatives), phosphatidyl serine complex, phosphatidyl choline, cobalamin (vitamin B12), intrinsic factor, pyridoxine (vitamin B6 or its derivatives), thiamine (vitamin B1), biotin (vitamin B7), niacin (vitamin B3), riboflavin (vitamin B2), n-acetyl cysteine (NAC), sulphorophance, SAMe, RNA nucleotides, amino acids, glycine, GABA, CoQ10, L-Carnitine (or its derivatives), alpha lipoic acid, resveratrol, glutathione (or its derivatives), DIM (diindolylmethane), and TMG. Other areas that may be considered to restore health include management of an overburden of heavy metals, as determined by a urine provocation laboratory or management of amino acid deficiency determined through an examination. Corrective steps would be taken to either lower the heavy metals in the patient or supplementing the deficient amino acids.

Along with the breast wellness regimen 22, patients should conduct periodic DNA oxidative stress and/or estrogen-DNA adduct screening tests and thermography screening to determine effectiveness of the breast wellness regimen 22. Through monitoring, the effectiveness of the regimen 22 is observed to reduce inflammation and the associate risk of developing cancerous cells. The observation of the progress motivates and encourages the patient to continue and maintain the regimen.

The DNA oxidative stress screening test and estrogen-DNA adduct screening test may both be conducted in conjunction with the thermography screening. Alternatively, either one of the DNA oxidative stress and/or estrogen-DNA adduct screening tests may be conducted in conjunction with the thermography screenings.

Additionally, in the method above, the thermography tests are conducted in a series to detect changes. However, a single thermography test may be used to detect significant changes.

The above description is given by way of example, and not limitation. Given the above disclosure, one skilled in the art could devise variations that are within the scope and spirit of the invention disclosed herein, including various ways of testing for the depurinating estrogen-DNA adducts. Further, the various features of the embodiments disclosed herein can be used alone, or in varying combinations with each other and are not intended to be limited to the specific combination described herein. Thus, the scope of the claims is not to be limited by the illustrated embodiments.

1. A method for identifying a patient with an increased propensity to breast cancer and implementing a responsive treatment, the method comprising the steps of:
conducting at least one DNA oxidative stress, estrogen-DNA adduct screening test or a combination thereof on a patient to detect a first biomarker for detection of breast cancer;
conducting a thermography screening test on the patient to detect for a second biomarker for detection of breast cancer risk;
determining whether the first biomarker is positive;
determining whether the second biomarker is positive;
determining whether the patient is at a higher risk for breast cancer, wherein the patient is at a higher risk for breast cancer when the first and second biomarkers are positive;
producing a breast wellness regimen for the patient as a responsive treatment; and
implementing the breast wellness regimen.

2. The method of claim 1, wherein the DNA oxidative stress screening test detects high levels of DNA oxidative stress markers.

3. The method of claim 1, wherein the estrogen-DNA adduct screening test detects a level of depurinating estrogen-DNA adducts.

4. The method of claim 1, wherein the estrogen-DNA adduct screening test detects high ratios of depurinating estrogen-DNA adducts to their respective estrogen metabolites and conjugates.

5. The method of claim 1, wherein the first biomarker is positive when the ratios are high compared to females with a low risk for breast cancer.

6. The method of claim 1, wherein the conducting the thermography screening test comprises the steps of:
conducting a single thermography screening test on the patient to identify indications of area(s) of inflammation; or the steps of:
conducting a first thermography screening test on the patient to acquire a first heat map of the breasts of the patient indicating areas of normal to inflamed breast tissue;
conducting a second thermography screening test on the patient to acquire a second heat map of the breasts of the patient indicating areas of normal to inflamed breast tissue;
comparing the first and second heat maps to detect changes in inflammation and blood flow.

7. The method of claim 6, wherein the second biomarker is positive when there is a remarkable heat pattern in the first thermogram and/or significant difference between succeeding thermograms.

8. The method of claim 1, wherein the conducting the DNA oxidative stress, estrogen-DNA adduct screening test or a combination thereof is conducted in conjunction with a series of thermography screening tests resulting in a positive second biomarker.

9. The method of claim 1, wherein conducting the thermography screening test is performed if the first biomarker from the DNA oxidative stress, estrogen-DNA adduct screening test or a combination thereof is positive.

10. The method of claim 1, wherein the steps of conducting the DNA oxidative stress, estrogen-DNA adduct screening test or a combination thereof and conducting the thermography screening test are performed at regular intervals as the patient conducts the breast wellness regimen.

11. The method of claim 1, further comprising the step of conducting a series of thermography screenings at regular intervals as the patient conducts the breast wellness regimen to track efficacy of the breast wellness program.

12. The method of claim 1, wherein the breast wellness regimen includes the step of inducing lymphatic fluid flow through the patient's lymphatic system.

13. The method of claim 12, wherein the inducing step includes one or more of regular physical exercise, consumption of water, fitted clothing and lymphatic breast massage.

14. The method of claim 1, wherein the breast wellness regimen includes the step of improving at least one of the patient's immune balance, hormone balance, food intake, evaluation of the patient's digestion sufficiency, and toxin and waste elimination.

15. The method of claim 1, wherein the breast wellness regimen includes prescribing estrogen detoxification supplements to the patient.

16. The method of claim 15, wherein the supplements includes at least one compound comprising diindolylmethane, Nrf2 activators, SAMe (S-adenosyl methionine), TMG (trimethylglycine), resveratrol, NAC (Nacetylcysteine), folic acid, glutathione or a combination thereof.

17. The method of claim 1, further comprising conducting a metabolic profile.

18. The method of claim 17, wherein the results of the metabolic profile are used for directed genetic testing.

19. The method of claim 18, wherein a targeted supplement is prescribed to the patient based upon results of the directed genetic testing.

20. The method of claim 19, wherein the targeted supplement is at least one compound comprising tetrahydrofolate, folate, folic acid (and its derivatives), phosphatidyl serine complex, phosphatidyl choline, cobalamin (vitamin B12), intrinsic factor, pyridoxine (vitamin B6 or its derivatives), thiamine (vitamin B1), biotin (vitamin B7), niacin (vitamin B3), riboflavin (vitamin B2), n-acetyl cysteine (NAC), sulphorophance, SAMe, RNA nucleotides, amino acids, glycine, GABA, CoQ10, L-Carnitine (or its derivatives), alpha lipoic acid, resveratrol, glutathione (or its derivatives), DIM (diindolylmethane), and TMG or a combination thereof.
